(19) United States
(12) Patent Application Publication
(10) Pub. No.: US 2026/0348746 A1
(43) Pub. Date: Nov. 5, 2026

(54) System for Measuring and Reporting Soil Moisture, Soil Temperature, and Sunlight Levels

(71) Applicant: Harvest Thyme, LLC, Maryville, TN (US)

(72) Inventor: Brian D. Canfield, Knoxville, TN (US)

(73) Assignee: Harvest Thyme, LLC, Maryville, TN (US)

(21) Appl. No.: 19/195,999

(22) Filed: May 1, 2025

(51) Int. Cl. G01D 21/02

(52) U.S. Cl. CPC G01D 21/02

(57) ABSTRACT

A system for measuring and reporting soil moisture, soil temperature, and sunlight levels includes a hub module connected to a gardener's WiFi network and sensor modules that are in radio communication with the hub module. The sensor modules may be planted in the gardener's yard, ground, planter/container, raised bed, or hanging baskets. The sensor modules can be moved to various locations from one season to the next. Data collected by the sensor modules are sent via radio to the hub module, which in turn sends that data to a webserver backend. The collected data are assimilated, analyzed along with information from a custom plant database, and presented to the gardener in an easy-to-read format via a mobile application running on a mobile computing device.

### FIELD

This invention relates to the field of monitoring conditions for growing plants. More particularly, this invention relates to a system for measuring and reporting soil moisture, soil temperature, and sunlight levels to a gardener.

### BACKGROUND

Many home gardeners lack specific information about the condition of the soil in which they are attempting to grow plants and about the levels of sunlight that is received by the plants. Even if soil and sunlight conditions are known, many gardeners do know exactly what soil and sunlight conditions are most conducive for growing the particular plants in their garden. This can be frustrating for many gardeners, in particular the beginner gardeners.

What is needed, therefore, is a system for measuring and reporting soil moisture, soil temperature, and sunlight levels to a gardener, and providing information to the gardener regarding whether the soil and sunlight conditions are appropriate for the plants the gardener wishes to grow.

### SUMMARY

The above and other needs are met by a system for measuring and reporting soil moisture, soil temperature, and sunlight levels. Embodiments of the system include a hub module (connected to a gardener's WiFi network) and sensor modules that are in radio communication with the hub module. The sensor modules may be planted in the gardener's yard, ground, planter/container, raised bed, or hanging baskets. The sensor modules can be moved to various locations from one season to the next. Data collected by the sensor modules are sent via radio to the hub module, which in turn sends that data to a webserver backend. Finally, the collected data are assimilated, analyzed along with information from a custom plant database, and presented to the gardener in an easy-to-read format via a mobile application running on a mobile computing device.

In preferred embodiments, the sensor modules are 11 inches long, 7.5 inches of which is buried in the soil. Every 20 minutes, the sensor modules send an average moisture value, current soil temperature, and average sunlight value to the hub module for data collection. When data is to be collected, the hub module sends a “wakeup” signal to all of the sensor modules. The hub module then sends a “harvest” signal to retrieve specific data from each sensor module. As each specific sensor module's data are received, the hub module sends that specific sensor module's data to the webserver backend, then proceeds to retrieve the next sensor module's data. After all the sensor module data are collected and sent to the webserver backend, the hub module sends a “sleep” signal to the sensor modules to put them in a hibernation mode. In a preferred embodiment, the sensor modules will report 72 data points for moisture, temperature, and sunlight over the course of 24 hours.

The sensor modules are designed to “wake up” automatically every five minutes to sample the soil moisture, soil temperature, and sunlight-then go back to sleep. This is to preserve battery life and accumulate average values for moisture and sunlight. The sensor modules broadcast data in the 2.4 GHz band on a specified channel to the hub module. In addition to the 2.4 GHz radio for sensor module communication, the hub module has a built-in WiFi chip for connecting to the gardener's local WiFi network in the 2.4 GHz band on a different channel than the one used for sensor module communication to minimize radio interference. Each sensor module is given a specific address during the sensor module pairing process. This allows for daisy-chaining sensor modules together to maximize distance between sensor modules and the hub module. Preferred embodiments use an addressing topology by which data can be sent through up to five sensor modules deep, and across five sensor modules locally, thereby providing the ability to connect up to 3125 sensor modules to one hub module.

The mobile application running on the mobile computing device takes information from the sensor modules and interprets the information with reference to a custom plant database. Based on this, the mobile application informs the gardener about specific watering and sunlight needs, and provides guidance for growing the gardener's specific plants given the current weather and soil/light/temperature conditions.

The gardener “plants” a sensor module in their yard, garden, hanging basket, etc., and the area associated with that sensor module is referred to as a “Zone.” The mobile application takes data collected from the sensor module, compares it to the requirements of the plants in that “Zone,” and reports back to the gardener whether the soil is too wet or too dry, whether the soil temperature is too low or too high, and whether the amount and duration of sunlight is appropriate for the plants in that “Zone.” The moisture, sunlight, and temperature information is logged and can be viewed by the gardener by the hour, the day, the week, or the month as an aggregate value. In some embodiments, the mobile application also instructs the gardener on best practices for seedling growth, fertilizing, promoting soil health, and preventing garden pests.

Accordingly, some preferred embodiments are directed to an apparatus for measuring and reporting soil conditions and sunlight levels in a plant growing area in which plants are maintained by a gardener. The apparatus comprises one or more sensor modules, a hub module, a webserver backend, and a mobile application running on a mobile computing device operated by the gardener.

Each of the sensor modules include a soil temperature sensor, a soil moisture sensor, a light sensor, a first microcontroller, a first wireless transmission and reception device, a battery, and a solar panel, all of which are disposed on or within a single weatherproof housing. The soil temperature sensor is operable to measure soil temperature within the plant growing area and generate a soil temperature signal based thereon. The soil moisture sensor is operable to measure soil moisture within the plant growing area and generate a soil moisture signal based thereon. The light sensor is operable to measure light levels within the plant growing area and generate a light level signal based thereon. The first microcontroller is operable to periodically generate soil condition data based on one or more of the soil temperature signal, the soil moisture signal, and the light level signal. The first wireless transmission and reception device that is operable to wirelessly transmit one or more wireless soil data signals that include the soil condition data. The first wireless transmission and reception device is also operable to wirelessly receive hub signals transmitted from the hub module. The battery provides power to one or more of the soil temperature sensor, soil moisture sensor, light sensor, first microcontroller, and first wireless transmission and reception device, and the solar panel generates electrical power to charge the battery.

The hub module includes a microcontroller that is operable to periodically:
generate wakeup signals that cause the one or more sensor modules to initiate measurements of soil temperature, soil moisture, and light levels,
generate retrieve data signals (also referred to herein as “Harvest” signals) that cause the one or more sensor modules to transmit the one or more wireless soil data signals, and
send at least the soil condition data to the webserver backend via the internet.

The hub module also includes a wireless transmission and reception device that is operable to:
transmit the wakeup signals and the retrieve data signals to the one or more sensor modules, and
receive the one or more wireless soil data signals transmitted from the one or more sensor modules; and

The webserver backend includes one or more computer processors that are operable to execute instructions to:
receive the soil condition data via the internet;
receive and maintain information regarding the one or more sensor modules, hub module, the gardener, the plant growing area, and the plants in the plant growing area; and
provide access to the soil condition data and other information maintained on the webserver backend to a mobile application; and

The mobile application includes instructions that cause the mobile computing device to:
access the soil condition data from the webserver backend;
access plant information from a plant database regarding the types of plants within the plant growing area;
access weather information from a weather application programming interface; and
based on the weather information, soil condition data, and plant information, generate information for display on the mobile computing device to advise the gardener about watering and sunlight needs of specific plants within the plant growing area.

In some embodiments, each of the sensor modules is operable to:
awaken from a sleep mode at X minute intervals, acquire measurements of soil temperature, soil moisture, and light levels, generate soil condition data based on the acquired measurements, and return to the sleep mode; and
awaken from the sleep mode at Y minute intervals and transmit the soil condition data.

In some embodiments, X is 5 and Y is 18.

In some embodiments, each sensor module is paired in a wireless network with the hub module, and the hub module may be paired in the wireless network with one or more sensor modules.

In some embodiments, each of the sensor modules is operable to awaken from a sleep mode upon receipt of a wakeup signal transmitted from the hub module with which the sensor module is paired.

In some embodiments, each of the sensor modules is operable to transmit the soil condition data upon receipt of a retrieve data (Harvest) signal transmitted from the hub module with which the sensor module is paired.

In some embodiments, the hub module receives the soil condition data sequentially from each of the sensor modules with which the hub module is paired, sends the received soil condition data to the webserver backend via the internet, and sends a sleep signal to each of the sensor modules with which the hub module is paired.

### BRIEF DESCRIPTION OF THE DRAWINGS

Other embodiments of the invention will become apparent by reference to the detailed description in conjunction with the figures, wherein elements are not to scale so as to more clearly show the details, wherein like reference numbers indicate like elements throughout the several views, and wherein:

FIG. 1 depicts a functional block diagram of a system for measuring and reporting soil moisture, soil temperature, and sunlight levels according to an embodiment of the invention;

FIG. 2 depicts a top perspective view of a sensor module of a system for measuring and reporting soil moisture, soil temperature, and sunlight levels according to an embodiment of the invention;

FIG. 3 depicts a top plan view of the sensor module of FIG. 2;

FIG. 4 depicts a front elevation view of the sensor module of FIG. 2;

FIG. 5 depicts a side elevation view of the sensor module of FIG. 2;

FIG. 6 depicts a functional block diagram of a webserver backend, a mobile application, and weather application programming interface (API) of a system for measuring and reporting soil moisture, soil temperature, and sunlight levels according to an embodiment of the invention;

FIG. 7 depicts an operational flow diagram of a system for measuring and reporting soil moisture, soil temperature, and sunlight levels in a normal operational mode according to an embodiment of the invention;

FIGS. 8A-8B depict an exemplary display screen of a gardener's mobile computing device showing a tomato plant profile and indicating a particular Zone meets moisture and sunlight requirements for growing tomatoes;

FIGS. 9A-9B depict exemplary display screens of a gardener's mobile computing device showing Moisture Profile data for a particular week and month;

FIGS. 10A-10B depict exemplary display screens of a gardener's mobile computing device showing weather data generated by a Weather API and sunlight data for a particular day;

FIG. 11 depicts an operational flow diagram of a system for measuring and reporting soil moisture, soil temperature, and sunlight levels in a pairing mode according to an embodiment of the invention; and

FIGS. 12-14 depict examples of a sensor network topography of a system for measuring and reporting soil moisture, soil temperature, and sunlight levels according to an embodiment of the invention.

### DETAILED DESCRIPTION

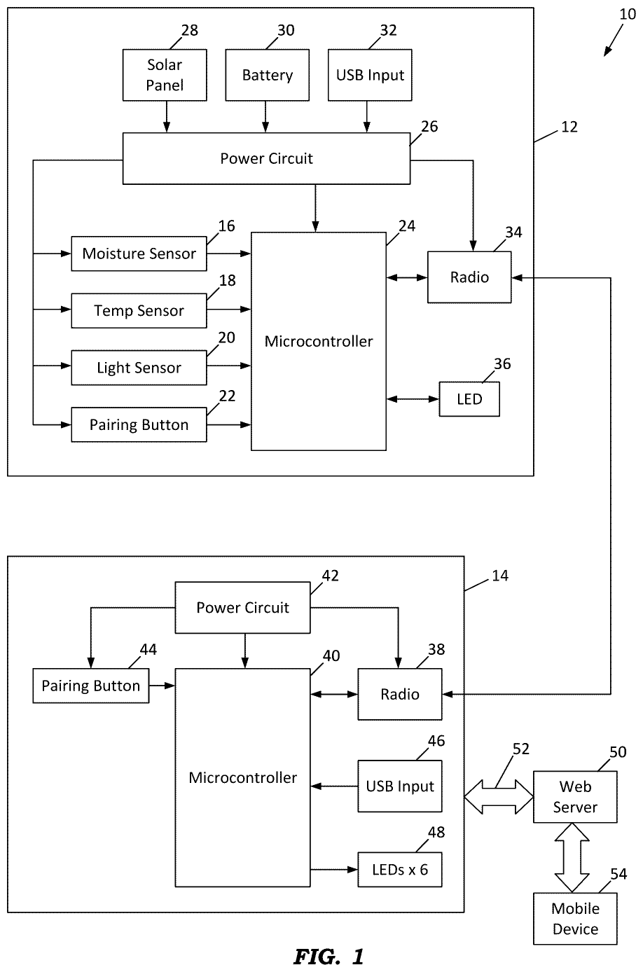

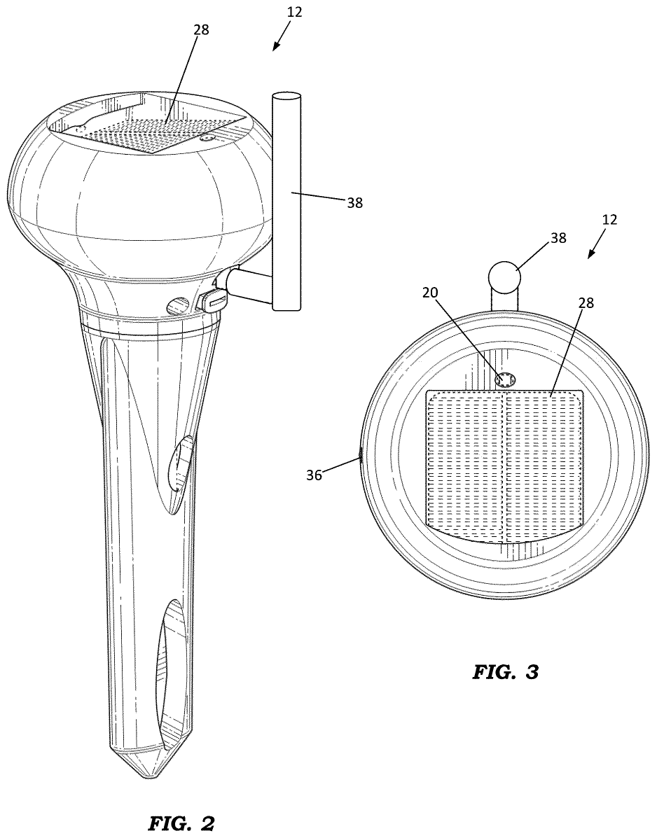

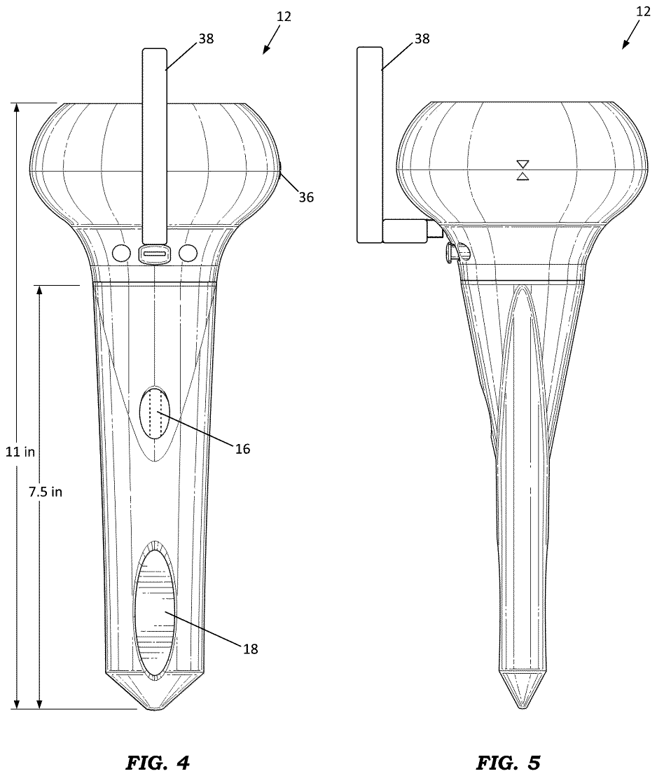

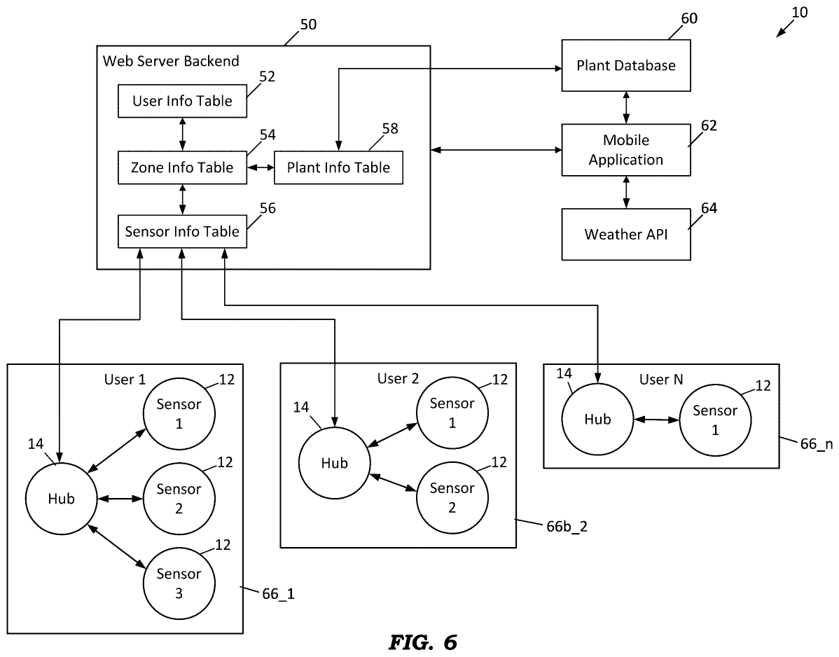

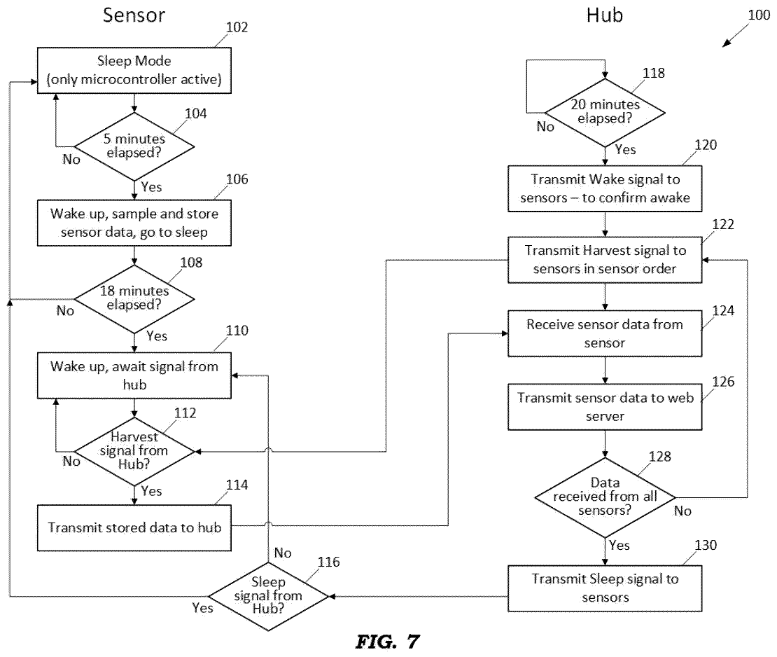

As depicted in FIG. 1, a preferred embodiment of a system for measuring and reporting soil moisture, soil temperature, and sunlight levels 10 includes at least one sensor module 12 that is in wireless communication with at least one hub module 14. The sensor module 12 includes a soil moisture sensor 18, a soil temperature sensor 16, and a light sensor 20, each of which provides a sensor signal to a microcontroller 24. In one embodiment, the moisture sensor 18 is a model number Hw-390 capacitive soil sensor module, the temperature sensor 16 is a model number DS18B20 manufactured by HiLetgo, and the light sensor 20 is a photoresistor having model number EBOOT-RESISTOR-05 manufactured by EBOOT, and the microcontroller 24 is a model number ATMega328P manufactured by Microchip Technology Incorporated. Power for the sensor module 12 is provided by a battery 30, and the power is distributed by a power circuit 26. During normal use, the battery 30 is charged by a solar panel 28. The battery 30 may also be charged via a USB input 32. The sensor module 12 includes a radio module 34 that provides for two-way wireless communication between the sensor module 12 and the hub module 14. In a preferred embodiment, the radio module 34 is a model number nRF24L01+ manufactured by Nordic Semiconductor that operates in the 2.4 GHz frequency band. The sensor module 12 also includes a pairing button 22 that is operated by a gardener during pairing operations as described in more detail hereinafter. A status LED 36 provides status information to the gardener. In the preferred embodiment, the sensors 16, 18, and 20, microcontroller 24, battery 30, solar panel 28, USB input 32, power circuit 26, pairing button 22, radio module 34, antenna 38, and LED 36 are all disposed on or within a single weatherproof housing as depicted in FIGS. 2-5.

As the term is used herein, the “gardener” is a person responsible for planting and maintaining the plants within the planting zones. The gardener may also be referred to herein as the “user” of the system 10.

A preferred embodiment of the hub module 14 includes a microcontroller 40 (model number ESP8266 manufactured by Espressif Systems) that communicates with the sensor module 12 via a radio module 38 (model number nRF24L01+ manufactured by Nordic Semiconductor). Power for the hub module 14 is provided by an AC/DC USB converter that plugs into a USB input 46, which power is distributed by a power circuit 42. A preferred embodiment of the hub module 14 also includes six LED's 48 that provide status information to the gardener, and a pairing button 44 that is operated by the gardener during pairing operations as described in more detail hereinafter.

Referring now to FIGS. 2-5, a preferred embodiment of a housing of the sensor module 12 is depicted. The upper bulbous portion of the housing of the sensor module 12 houses the various electrical components described above. The solar panel 28 and the light sensor 20 are preferably disposed on the top surface of the module 12 in an optimal position for receiving sunlight. The moisture sensor 18 and temperature sensor 16 are disposed on the shaft portion of the module 12 that is designed for insertion into the soil. The pairing button 22 is preferably located on a circuit board inside the weatherproof housing. The LED 36 is also housed within the weatherproof housing and is visible at the top of the housing. A preferred embodiment of the sensor module 12 also includes an antenna 38 extending from the side of the upper bulbous portion of the housing.

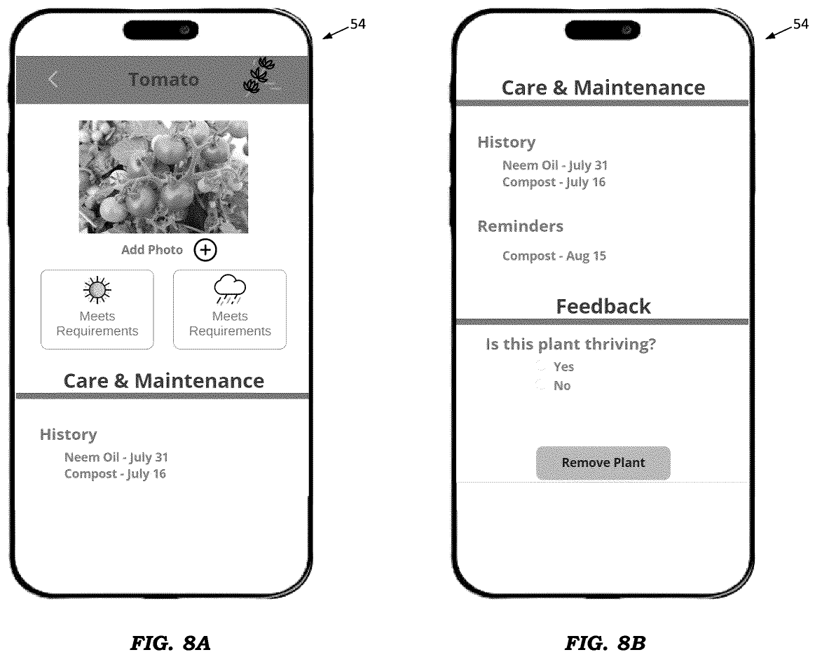

Referring now to FIG. 6, operations of the system 10 involving the webserver backend 50 are depicted. The webserver backend 50 includes a User Information Table 52, a Zone Information Table 54, a Sensor Information Table 56, and a Plant Information table as described below.
The User Information Table 52 includes the user's name, username, email, address, city, state, zip code, hub MAC address, list of active zones, and unique user ID code autogenerated by the webserver backend 50.
The Zone Information Table 54 includes Zone name, address, color, blink frequency, watering profile (self-determined), sunlight profile (self-determined), sun timing (self-determined) for morning or afternoon sunlight received, and a unique zone ID code autogenerated by the webserver backend 50. (This table relates to the User table via the unique user ID code.)
The Sensor Information Table 56 includes a sensor unique ID (autogenerated by the webserver backend 50), sensor address, MAC address of the hub module 14 that the sensor module 12 is paired to, moisture data field, sunlight data field, temperature data field, solar power data field, battery power data field, and watering event (self-determined within the sensor). (This table relates to the User Information Table via the user ID code and to the Zone Table via the Zone ID code.)
The Plant Information Table 58 includes Plant Name, Plant Image, Plant Type, USDA Zone in which the plant thrives, Max height (inches), Avg height (inches), Plant Life Cycle (Annual, Perennial, etc.), and a unique plant ID code autogenerated by the webserver backend 50. This table relates to the Zone Table through a Zone-Plant table where the zone ID is paired with the plant ID as the user assigns a plant to a Zone. Additionally, the plant ID is used to retrieve other information from the Plant Database using the plant ID code.

The Zone Information Table 54 is in communication with the other tables 52, 56, and 58. The Plant Information Table 58 is also in communication with the Plant Database 60. The Mobile Application 62 (running on the gardener's mobile computing device 54—see FIG. 1) is in communication with the webserver backend 50, the Plant Database 60, and a weather API 64.

In the exemplary embodiment of FIG. 6, there are N number of users and N number of user Zones, including Zones 66_1, 66_2, and 66_n. Each User can potentially have up to Z number of Zones, where Z<3125. Each Zone includes at least one hub module 14 and at least one sensor module 12. Each sensor module 12 needs at least one hub module 14 to communicate with, but the hub module 14 can communicate with Z number of sensor modules 12 (all for the same User). Thus, each sensor module 12 does not need its own dedicated hub module 14. The Sensor Information Table 56 of the webserver backend 50 is in communication with the hub module 14.

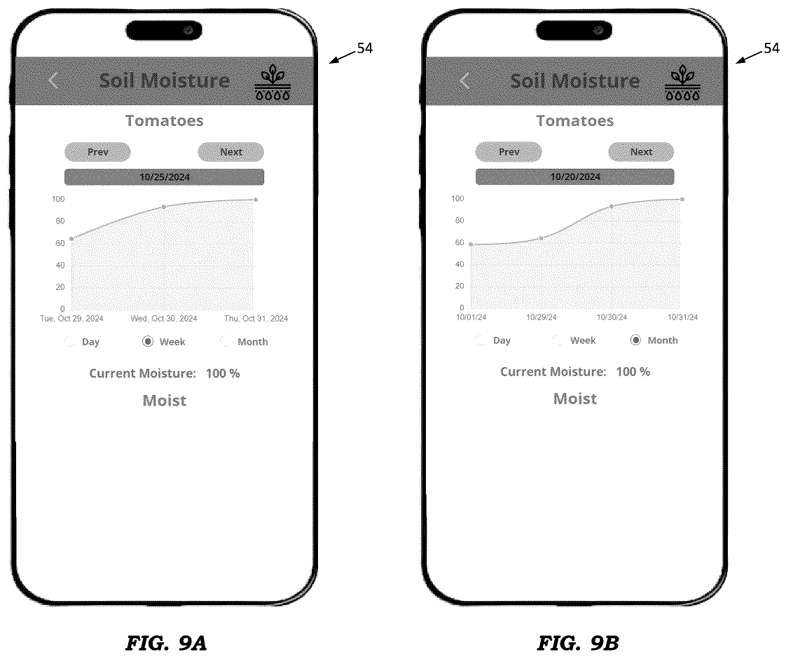

An operational process flow diagram of the system 10 is depicted in FIG. 7. As described in more detail hereinafter, some of the steps of the process 100 are performed by the microcontroller 24 of the sensor module 12 and some steps are performed by the microcontroller 40 of the hub module 14. In describing the system operation with reference to FIG. 7, it is assumed that the sensor module 12 and the hub module 14 have already been paired as described in more detail hereinafter.

When the sensor module 12 is operating in Sleep Mode (step 102), only the microcontroller 24 is active, with a clock in the microcontroller 24 measuring various intervals of time. Once five minutes have elapsed since Sleep Mode was first entered (step 104), the sensor module 12 “wakes up” to sample and store light, temperature, and moisture data in memory, and then goes back to into Sleep Mode (step 106). Step 106 is performed again at 10 minutes elapsed time and at 15 minutes elapsed time. Once 18 minutes have elapsed since Sleep Mode was first entered (step 108), the sensor module 12 wakes up and awaits a signal from the hub module 14 (step 110). Upon receipt of a Harvest signal from the hub module 14 (step 112), the sensor module 12 retrieves the light, temperature, and moisture data that have been measured and stored in its memory at the previous three data collection intervals, and transmits the retrieved data to the hub module 14 (step 114).

Meanwhile, a clock in the microcontroller 40 of the hub module 14 continuously measures 20 minute time intervals. Once 20 minutes have elapsed (step 118), the radio 38 of the hub module 14 transmits a Wake signal that is received by all sensor modules 12 that have been paired with the hub module 14. (At the 18-minute elapsed time, the sensor modules 12 should be awake and listening for the Wake signal from the hub module 14. If they are not awake, the Wake signal will wake them up.) The hub module 14 then transmits Harvest signals sequentially to all of the sensor modules 12 in order, wherein each Harvest signal is addressed to a particular one of the sensor modules 12. The hub module 14 then receives the measured data transmitted from each sensor module 12 (step 124) and transmits the data to the webserver backend 50 (step 126). The sensor data sent to the webserver backend 50 preferably includes moisture data, sunlight data, temperature data, watering event data, and power data. In a preferred embodiment, the watering event data is a binary 1 or 0 that indicates whether the sensor module 12 has detected a watering event based on a sudden, drastic change in moisture values. Once it is confirmed that the sensor data have been received from all of the paired sensor modules 12 (step 128), the hub module 14 transmits a Sleep signal to all of the sensor modules 12 (step 130).

Upon receipt of the Sleep signal at each sensor module 12 (step 116), the sensor module 12 reenters Sleep Mode (step 102), and the process repeats.

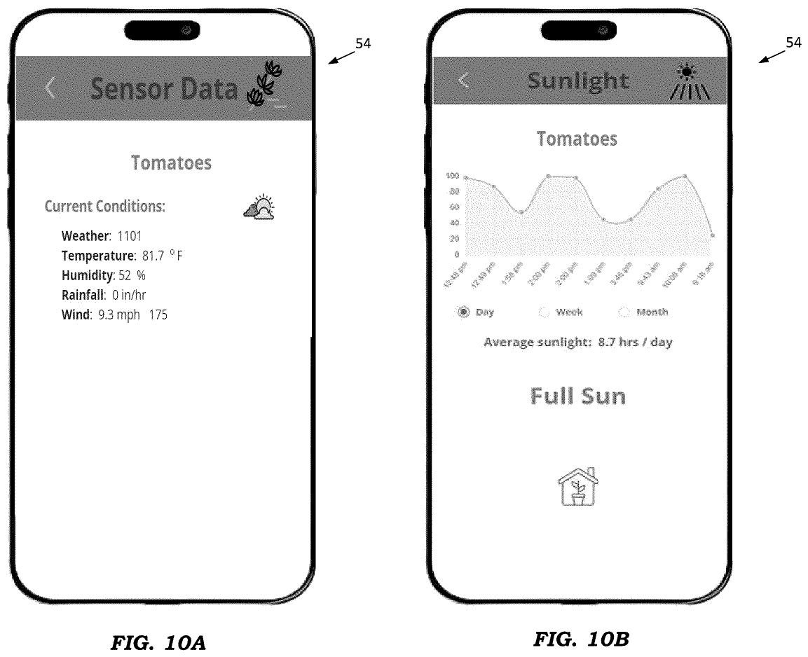

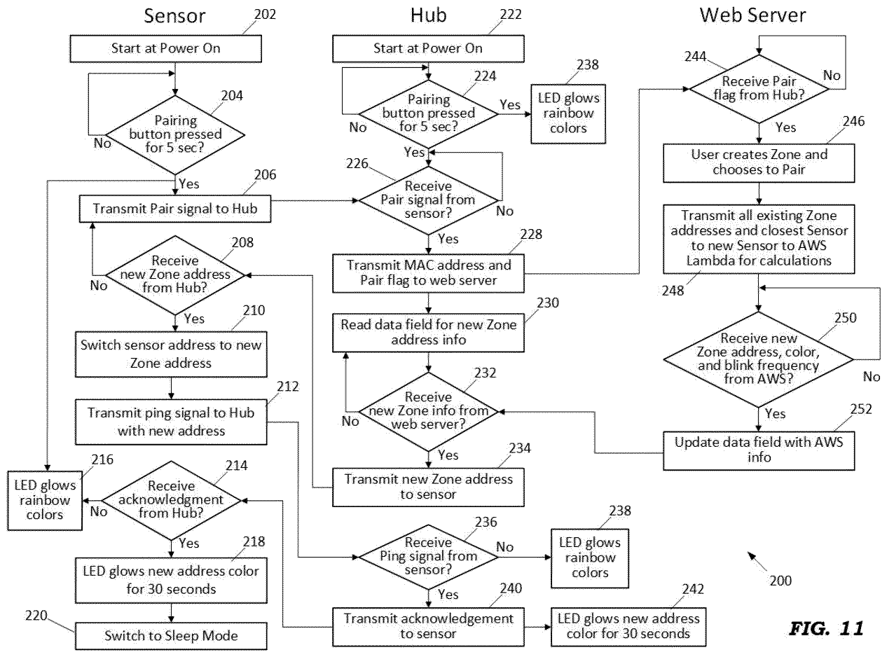

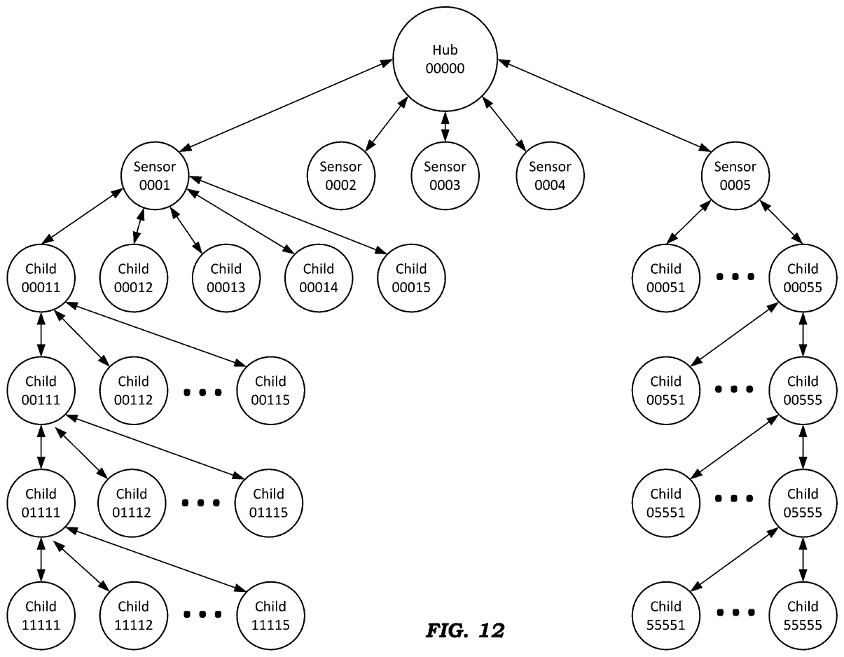

For purposes of describing the operation of the webserver backend 50 and the mobile application 62, an example will be described in which the plants in Zone 66_1 are tomatoes. With reference again to FIG. 6, the soil condition data transmitted from the hub module 14 to the webserver backend 50 are received in the Sensor Info Table 56. The mobile application 62 accesses the sensor data from the Sensor Info Table 56, and takes Moisture Detail information from the Plant Database 60 and determines based on the sensor data whether Zone 66_1 (tomatoes) meets moisture requirements. The mobile application 62 then reports whether Zone 66_1 (tomatoes) meets requirements for growing tomatoes. For example, FIGS. 8A-8B depict an example of the display screen of the gardener's mobile computing device 54 showing the tomato plant profile and indicating that the Zone 66_1 meets the requirements for moisture and sunlight for growing tomatoes. The mobile application 62 also provides advice regarding watering in Zone 66_1 based on the current sensor data, weather data from the Weather API, and Plant Moisture Detail from the Plant Database 60. FIG. 10 depicts an example of the display screen of the gardener's mobile computing device 54 showing the weather data generated by the Weather API. Also for example, the Plan Moisture Detail for tomatoes may be “Keep Moist (as opposed to Dry Between).” The gardener can also see Moisture Profile data for the day, week, and month. FIGS. 9A-9B depict an example of the display screen of the gardener's mobile computing device 54 showing the Moisture Profile data for a particular week (FIG. 9A) and month (FIG. 9B). FIG. 10B depicts an exemplary display screen showing sunlight data over a particular day within the same zone.

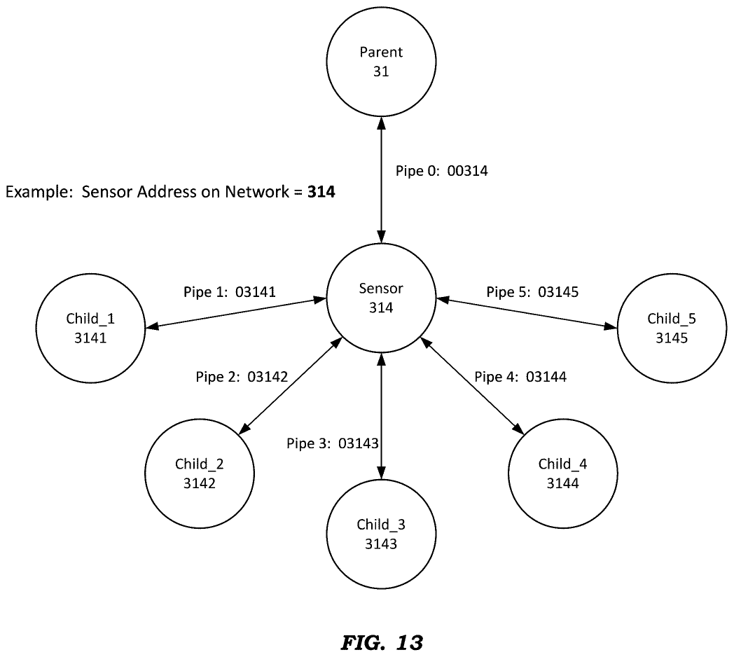

A pairing process flow diagram of the system 10 is depicted in FIG. 11. As described in more detail hereinafter, some of the steps of the process 200 are performed by the microcontroller 24 of the sensor module 12, some steps are performed by the microcontroller 40 of the hub module 14, and some are performed by the webserver backend 50. Once the sensor module 12 is powered on (step 202), the microcontroller 24 monitors the state of the pairing button 22 to detect whether it has been pressed. When the pairing button 22 is pressed for at least five seconds (step 204), a Pair signal is transmitted from the radio 34 of the sensor module 12 to the hub module 14 (step 206) and the LED 36 is activated to glow rainbow colors (step 216). The sensor module 12 then listens for a signal from the hub module 14.

Once the hub module 14 is powered on (step 222), the microcontroller 40 monitors the state of the pairing button 44 to detect whether it has been pressed. When the pairing button 44 is pressed for at least five seconds (step 224), the LED 48 is activated to glow rainbow colors (step 238), and the hub module 14 listens for a signal from the sensor module 12. Once the hub module 14 receives the Pair signal from the sensor module 12 (step 226), the radio 38 of the hub module 14 transmits its MAC address and a Pair flag to the webserver backend 50 via a communication network 52, such as the Internet (step 228). In a preferred embodiment, the Pair Flag is the value “55555” that is transmitted to the Sensor Data table. The webserver backend 50 workflows detect a change in data value, and if the new value is a “55555”, then it recognizes that as a Pairing Event and starts the pairing process on the webserver backend 50.

Once the webserver backend 50 receives the Pair flag from the hub module 14 (step 244), the webserver backend 50 begins the pairing process based on the gardener creating a Zone and choosing to Pair using the mobile application (step 246). The webserver backend 50 then transmits all existing Zone addresses, and the identity of the previously-paired sensor module that is closest to the new sensor module to the Amazon Web Service (AWS) “Lambda” for calculations (step 248). Once the webserver backend 50 receives the new Zone address, Zone address color, and blink frequency from AWS Lambda (step 250), the webserver backend 50 sends this information to the hub module 14 (step 252).

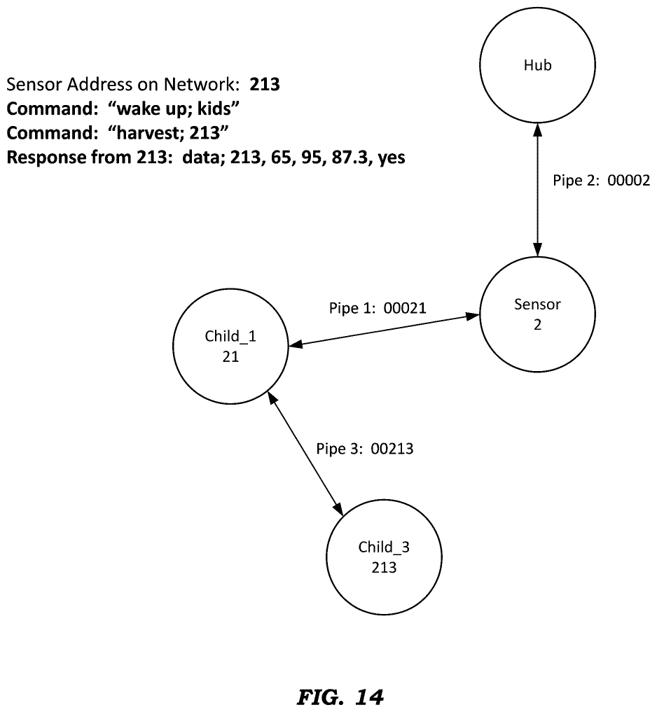

The Zone address color and blink frequency refer to the LEDs 48 on the hub module 14 and the LED 36 on top of the sensor module 12. Zone address colors are assigned to the sensor addresses based on their parent color. For example, with reference to FIG. 12, sensor modules 12 having a 00001 parent will have a red color LED, sensor modules 12 having a 00002 parent will have an orange LED, etc., with the final sensor module 12 whose parent is 00005 having a blue color LED. The blink frequency indicates how deep in the network topography the sensor module 12 resides-not blinking if at the parent level, blinking once for a first child, blinking twice if a second child (grandchild), etc. The Zone address colors and blink frequencies provide a visual confirmation and indication of which parent and location of depth of network topography to which each sensor module belongs.

In the meantime, microcontroller 24 of the hub module 14 has been periodically reading a particular data field to determine whether it has been updated with new Zone information from the webserver backend 50 (step 230). When new Zone information is detected (step 232), the hub module 14 transmits the new Zone address to the sensor module 12 via the radio 38 (step 234). When the sensor module 12 receives the new Zone address transmitted from the hub module 14 (step 208), the microcontroller 24 replaces the sensor module's address with the new Zone address (step 210), and transmits a ping signal containing the new Zone address back to the hub module 14 (step 212). Once the hub module 14 receives the ping signal from the sensor module 12 (step 236), the hub module 14 transmits an acknowledgment signal to the sensor module 12 (step 240) and the LED of the hub module glows the new Zone address color for 30 seconds (step 242). If pairing is successful, the sensor module 12, the hub module 14, and the mobile application all show the same color and blink at the blink frequency indicated.

Once the sensor module 12 receives the acknowledgment signal from the hub module 14 (step 214), the LED of the sensor module 12 glows the new Zone address color for 30 seconds (step 218) and the sensor module 12 switches to Sleep Mode (step 220).

FIGS. 12-14 depict examples of a sensor network topography implemented by a preferred embodiment of the system 10.

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. An apparatus for measuring and reporting soil conditions and sunlight levels in a plant growing area in which plants are maintained by a gardener, the apparatus comprising:
one or more sensor modules, each comprising:
a soil temperature sensor that is operable to measure soil temperature within the plant growing area and generate a soil temperature signal based thereon;
a soil moisture sensor that is operable to measure soil moisture within the plant growing area and generate a soil moisture signal based thereon;
a light sensor that is operable to measure light levels within the plant growing area and generate a light level signal based thereon;
a first microcontroller that is operable to periodically generate soil condition data based on one or more of the soil temperature signal, the soil moisture signal, and the light level signal;
a first wireless transmission and reception device that is operable to wirelessly transmit one or more wireless soil data signals that include the soil condition data, and operable to wirelessly receive hub signals transmitted from a hub module;
a battery for providing power to one or more of the soil temperature sensor, soil moisture sensor, light sensor, first microcontroller, and first wireless transmission and reception device; and
a solar panel for generating electrical power to charge the battery,
wherein the soil temperature sensor, soil moisture sensor, light sensor, first microcontroller, first wireless transmission and reception device, battery, and solar panel are all disposed on or within a single weatherproof housing;
a hub module comprising:
a second microcontroller that is operable to periodically
generate wakeup signals that cause the one or more sensor modules to initiate measurements of soil temperature, soil moisture, and light levels,
generate retrieve data signals that cause the one or more sensor modules to transmit the one or more wireless soil data signals, and
send at least the soil condition data to the webserver backend via the internet; and
a second wireless transmission and reception device that is operable to
transmit the wakeup signals and the retrieve data signals to the one or more sensor modules, and
receive the one or more wireless soil data signals transmitted from the one or more sensor modules; and
a webserver backend comprising one or more computer processors that are operable to execute instructions to:
receive the soil condition data via the internet;
receive and maintain information regarding the one or more sensor modules, the
hub module, the gardener, the plant growing area, and plants in the plant growing area; and
provide access to the soil condition data and other information maintained on the webserver backend to a mobile application; and
the mobile application running on a mobile computing device operated by the gardener, the mobile application comprising instructions that cause the mobile computing device to:
access the soil condition data from the webserver backend;
access plant information from a plant database regarding types of plants within the plant growing area;
access weather information from a weather application programming interface; and
based on the weather information, soil condition data, and plant information, generate information for display on the mobile computing device to advise the gardener about watering and sunlight needs of specific plants within the plant growing area.

2. The apparatus of claim 1 wherein each of the one or more sensor modules is operable to:
awaken from a sleep mode at X minute intervals, acquire measurements of soil temperature, soil moisture, and light levels, generate soil condition data based on the acquired measurements, and return to the sleep mode; and
awaken from the sleep mode at Y minute intervals and transmit the soil condition data.

3. The apparatus of claim 2 wherein X is 5 and Y is 18.

4. The apparatus of claim 1 wherein each of the one or more sensor modules is paired in a wireless network with the hub module, and wherein the hub module may be paired in the wireless network with one or more sensor modules.

5. The apparatus of claim 4 wherein each of the one or more sensor modules is operable to awaken from a sleep mode upon receipt of a wakeup signal transmitted from the hub module with which the sensor module is paired.

6. The apparatus of claim 4 wherein each of the one or more sensor modules is operable to transmit the soil condition data upon receipt of a retrieve data signal transmitted from the hub module with which the sensor module is paired.

7. The apparatus of claim 4 wherein the hub module receives the soil condition data sequentially from each of the one or more sensor modules with which the hub module is paired, sends the received soil condition data to the webserver backend via the internet, and sends a sleep signal to each of the one or more sensor modules with which the hub module is paired.
